(19) United States
(12) Patent Application Publication
(10) Pub. No.: US 2026/0325385 A1
(43) Pub. Date: Oct. 22, 2026

(54) SURGICAL CHAIR FOR MICROSCOPY

(71) Applicant: THE CATHOLIC UNIVERSITY OF KOREA INDUSTRY-ACADEMIC COOPERATION FOUNDATION, Seoul (KR)

(72) Inventors: Jae Sang OH, Seoul (KR); Tae Kyu LEE, Namyangju-si (KR)

(73) Assignee: THE CATHOLIC UNIVERSITY OF KOREA INDUSTRY-ACADEMIC COOPERATION FOUNDATION, Seoul (KR)

(21) Appl. No.: 19/468,145

(22) Filed: Feb. 3, 2026

(30) Foreign Application Priority Data
Mar. 5, 2025 (KR) 10-2025-0028262

(51) Int. Cl. A61G 15/12; A61G 15/08

(52) U.S. Cl. CPC A61G 15/12; A61G 15/08

(57) ABSTRACT

The present disclosure relates to a surgical chair for microscopy, comprising: a base; a housing mounted on an upper portion of the base; a lifting post inserted and installed inside the housing; a seat coupled to an upper portion of the lifting post and configured to allow a user to sit thereon; a pair of footrests on which both feet are placed; a footrest connection frame connecting the seat and the footrests; and a seat lifting module configured to lift and lower the lifting post to simultaneously lift and lower the seat and the pair of footrests.

### CROSS-REFERENCE TO RELATED APPLICATIONS

A claim for priority under 35 U.S.C. § 119 is made to Korean Patent Application No. 10-2025-0028262 filed on Mar. 5, 2025 in the Korean Intellectual Property Office, the entire contents of which are hereby incorporated by reference.

### BACKGROUND

### 1. Technical Field

The present disclosure relates to a surgical chair for microscopy.

### 2. Description of Related Art

A chair used by a surgeon or for operating on a patient in a hospital should be able to maintain a comfortable posture in consideration of the physical characteristics of the surgeon in accordance with various surgical or medical situations.

Particularly, since a more precise and stable surgical process is required for microscopy surgery, which takes a long time, the surgeon sits on a surgical chair for microscopy and simultaneously operates equipment such as a microscope and a bovie.

However, in the conventional surgical chair for microscopy, when operating a foot switch for operating the microscope and the bovie, not only is the interval between the seat where the surgeon sits and the foot switch wide, but also the size of the seat is very large and the backrest is fixed. Accordingly, there is a problem in that the surgeon has to endure a long-term surgery while sitting on the edge of the seat.

In addition, in a surgical environment using a microscope, while the patient's head is fixed, the surgeon must observe the surgical site through the microscope. In this process, since the position of the microscope is far from the patient, the surgeon must perform the surgery in an uncomfortable posture, which leads to problems such as decreased surgical convenience, increased surgeon fatigue, and decreased surgical concentration.

### SUMMARY

The present disclosure is directed to solving the above-described problems, and an object of the present disclosure is to provide a surgical chair for microscopy that allows a surgeon to sit in a desired posture according to surgical situations and physical conditions, thereby performing surgery in a stable posture to improve surgical convenience, reduce surgeon fatigue, and enhance surgical concentration.

The problems to be solved by the present disclosure are not limited to the above-mentioned problems, and other problems not mentioned will be clearly understood by those skilled in the art from the following description.

The object of the present disclosure may be achieved by a surgical chair for microscopy, comprising: a base; a housing mounted on an upper portion of the base; a lifting post installed inside the housing; a seat coupled to an upper portion of the lifting post and configured to allow a user to sit thereon; a pair of footrests on which both feet are placed; a footrest connection frame connecting the seat and the footrests; and a seat lifting module configured to lift and lower the lifting post to simultaneously lift and lower the seat and the pair of footrests.

Here, the seat lifting module may include: a seat lifting driver disposed inside the housing to lift and lower the lifting post; and an operation pedal installed on the base to operate the seat lifting driver.

Further, the footrests may be rotatably coupled to the footrest connection frame such that an arrangement angle of the footrests relative to the seat is adjusted.

The surgical chair may include a pair of armrests on which both arms are placed; and an armrest adjuster configured to swivel and lift the armrests relative to the seat.

The armrest adjuster may include: a seat frame supported by the seat; an armrest bracket to which the armrests are tiltably coupled by a hinge member; a link having one end connected to the seat frame and another end connected to the armrest bracket; a connection joint provided on the seat frame, the link being coupled to the connection joint to be swivellable and liftable; an armrest driver supported on the armrest bracket to lift and lower the link relative to the seat frame; and an operation lever configured to manipulate an operation of the armrest driver.

The surgical chair may further include: a backrest configured to support a back of a surgeon sitting on the seat; a backrest frame supporting the backrest; a backrest bracket to which the backrest frame is coupled; and a seat bracket connecting the backrest bracket and the seat.

The surgical chair may include a hinge pin rotatably coupling the backrest bracket and the seat bracket such that the backrest is tiltable relative to the seat.

The surgical chair may further include: a plurality of coupling holes formed at intervals along a longitudinal direction of the backrest frame; and a coupling protrusion provided on the backrest bracket and configured to be coupled to any one of the plurality of coupling holes, wherein a height of the backrest is adjustable relative to the seat.

The surgical chair may further include an anti-slip pad disposed on the footrests to prevent slipping.

The surgical chair may include a plurality of casters rollably provided on the base to move the base along a floor.

### BRIEF DESCRIPTION OF THE FIGURES

FIG. 1 is a perspective view of a surgical chair for microscopy according to an embodiment of the present disclosure.

FIG. 2 is a rear perspective view of FIG. 1.

FIG. 3 is a bottom perspective view of FIG. 1.

FIG. 4 is a right side view of FIG. 1.

FIG. 5 is a plan view of FIG. 1.

FIG. 6 is an enlarged perspective view of a main part of the armrest adjuster of FIG. 1.

FIG. 7 is an exploded perspective view of FIG. 6.

FIG. 8 is a view showing an adjusted state of a footrest of the surgical chair for microscopy according to an embodiment of the present disclosure.

### DETAILED DESCRIPTION

Advantages and features of the present disclosure and methods for achieving them will be clarified with reference to the embodiments described below in detail together with the accompanying drawings. However, the present disclosure is not limited to the embodiments disclosed below but may be implemented in various different forms. These embodiments are merely provided to complete the disclosure of the present disclosure and to fully inform those skilled in the art of the scope of the present disclosure.

The terminology used herein is for describing the embodiments and is not intended to limit the present disclosure. In this specification, the singular form also includes the plural form unless otherwise specified in the phrase. “Comprises” and/or “comprising” used in the specification do not exclude the presence or addition of one or more other components in addition to the mentioned components. Like reference numerals refer to like components throughout the specification, and “and/or” includes each and every combination of one or more of the mentioned components. Although “first,” “second,” etc. are used to describe various components, it goes without saying that these components are not limited by these terms. These terms are only used to distinguish one component from another. Therefore, it goes without saying that a first component mentioned below may be a second component within the technical spirit of the present disclosure.

Unless otherwise defined, all terms (including technical and scientific terms) used herein may be used with meanings commonly understood by those skilled in the art to which the present disclosure belongs. Also, terms defined in commonly used dictionaries are not interpreted ideally or excessively unless explicitly and specially defined.

Hereinafter, the present disclosure will be described in detail with reference to the accompanying drawings

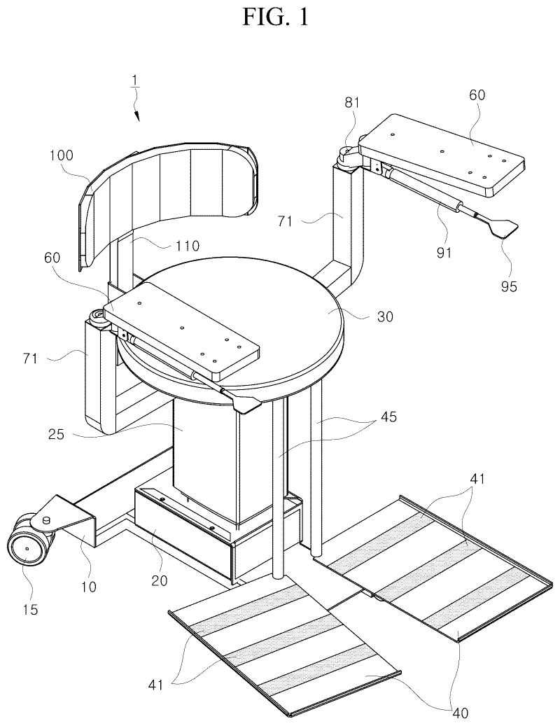

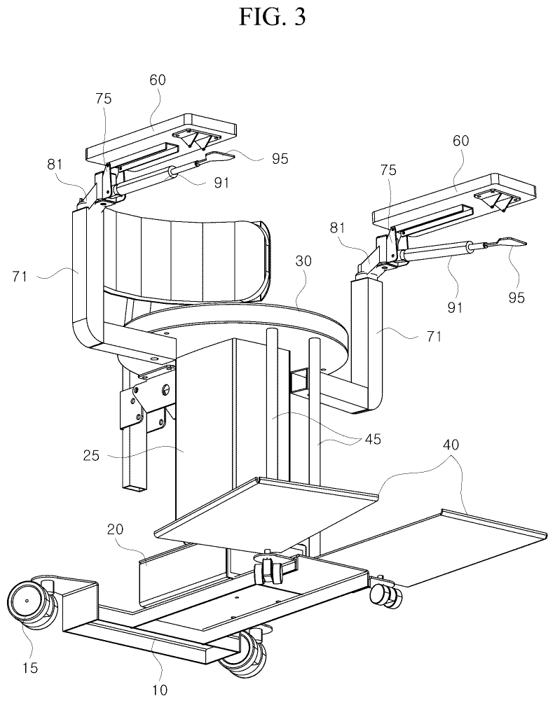

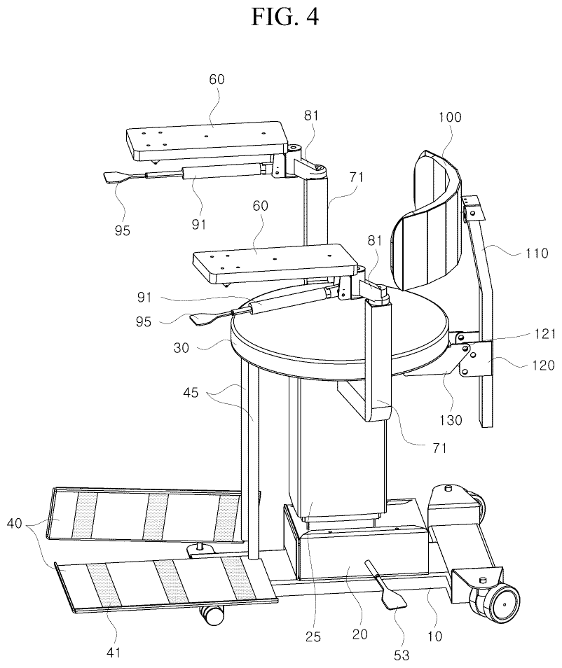

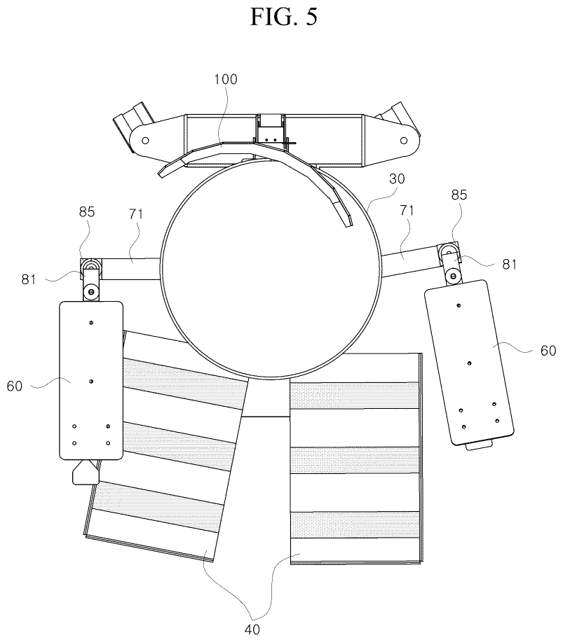

As shown in FIGS. 1 to 5, a surgical chair 1 for microscopy according to an embodiment of the present disclosure includes a base 10, a housing 20, a lifting post 25, a seat 30, footrests 40, a footrest connection frame 45, and a seat lifting module 50.

The base 10 is located below the housing 20 to support the housing 20.

On the lower portion of the base 10, a plurality of casters 15 are mounted, which are rollably provided on the base 10 to move the base 10 along the floor, that is, to move the surgical chair 1 for microscopy according to an embodiment of the present disclosure along the floor.

Some of the plurality of casters 15 may be provided with a locking pedal (not shown) for blocking the rotation of the wheels. For example, by stepping on one side of the locking pedal, the rotation of the wheels of the caster can be blocked, and by stepping on the other side of the locking pedal, the block can be released. This makes it possible to provide a safe surgical environment by preventing shaking or slipping during surgery.

In this way, by providing the plurality of casters 15, the position can be freely changed within the operating room, thereby improving surgical convenience.

The housing 20 spaces the seat 30 from the base and is installed upright on the upper portion of the base 10.

The lifting post 25 is formed to be long in the vertical direction.

A lower portion of the lifting post 25 is installed by being inserted into the housing 20, and an upper portion of the lifting post 25 is exposed to the outside of the housing 20.

The lifting post 25 is moved up and down, for example, lifted and lowered, by the seat lifting module 50.

The configuration of the seat lifting module 50 will be described later.

The seat 30 is coupled to the upper portion of the lifting post 25 so that a surgeon can sit thereon.

The seat 30 may be provided with a cushion member such as a sponge or foam resin therein to reduce fatigue and provide comfort even if the surgeon sits for a long time.

The pair of footrests 40 support both feet of the surgeon and have a flat rectangular plate shape.

The pair of footrests 40 are disposed between the base 10 and the seat 30.

An anti-slip pad 41 for preventing slipping may be provided on the footrests 40.

Here, although not shown, as an example, a foot switch may be seated on the footrest to perform microscopy surgery, and the footrest may have various sizes corresponding to the size of the foot switch placed on the footrest.

The footrest connection frame 45 is provided as a pair and has a rod shape of a predetermined length formed long in the vertical direction.

One end of the footrest connection frame 45 is connected to the bottom of the seat 30, and the other end is connected to the top of the footrest 40.

Accordingly, the seat 30 and the footrests 40 are integrally connected by the footrest connection frame 45.

Meanwhile, a hinge member (not shown), for example, a hinge pin, is provided at the lower end of each footrest connection frame 45, so that the pair of footrests 40 may be detachably and rotatably coupled. Here, each footrest 40 has a size that does not cause interference with the housing 20 and the lifting post 25 when the footrest 40 rotates.

In this way, as the footrest 40 is detachably coupled to the footrest connection frame 45, it is possible to selectively mount and use footrests 40 of various sizes corresponding to the size of the foot switch placed on the footrest 40.

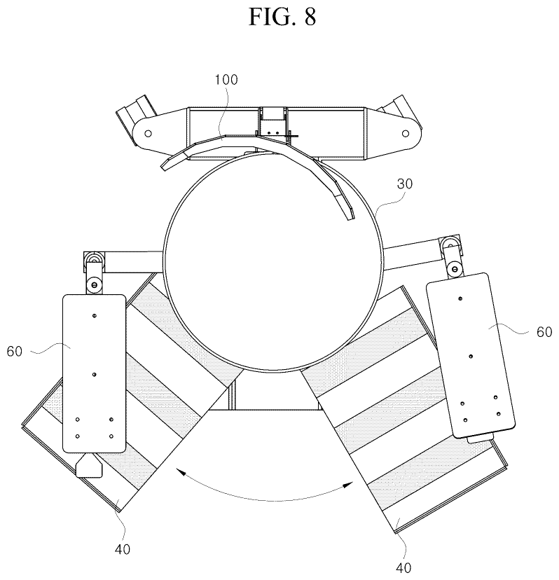

In addition, as the footrest 40 is rotatably coupled to the footrest connection frame 45, the arrangement angle of the pair of footrests 40 can be adjusted to a desired angle as shown in FIG. 8, so that the surgery can be performed while freely changing the position of the feet, and the angle of the footrest 40 can be adjusted according to the surgeon's ankle angle to minimize ankle fatigue and maintain a comfortable posture during surgery.

The seat lifting module 50 includes a seat lifting driver 51 installed inside the housing 20 to lift and lower the lifting post 25, and an operation pedal 53 installed on the base 10 to operate the seat lifting driver 51.

Here, as the seat lifting driver 51, a common gas spring in which a spring is accommodated inside a cylinder containing compressed gas, and the spring automatically provides restoring force and compressive force by compressing the gas inside the cylinder, for example, using a force by which the spring is automatically compressed or expanded by gas pressure, may be applied. Accordingly, according to the operation of the operation pedal 53 for manipulating the operation of the gas spring, the lifting post 25 can be raised or lowered using the restoring force or compressive force of the spring generated as the spring expands or compresses.

Also, although not shown, a motor and a lead screw may be applied as the seat lifting driver. In this case, various known structures may be applied to convert the rotational force of the motor into the vertical movement of the lifting post. As an example, the lifting post may be moved up and down by rotating a lead screw screw-coupled to the lower portion of the lifting post by a motor.

In this way, by simultaneously lifting and lowering the seat 30 and the footrests 40, the surgeon can sit in a desired posture according to the surgical situation and physical conditions, thereby performing surgery in a stable posture to improve surgical convenience, reduce surgeon fatigue, and enhance surgical concentration.

In addition, the surgical chair 1 for microscopy according to an embodiment of the present disclosure may include a pair of armrests 60 on which both arms are placed, and an armrest adjuster 70 for swiveling, lifting, and tilting the armrests 60 relative to the seat 30.

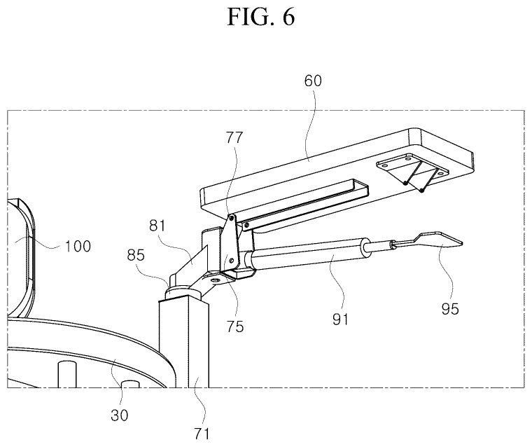

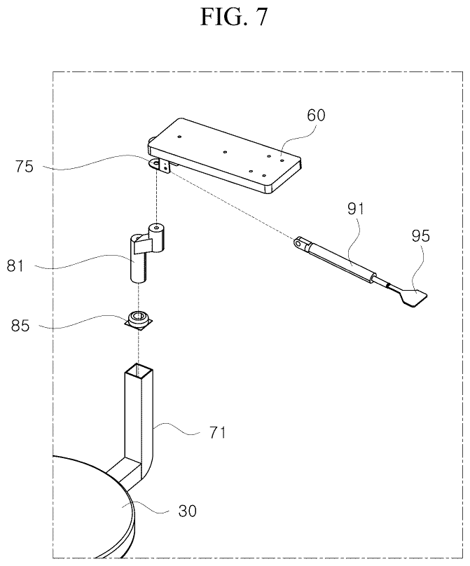

The armrest adjuster 70 includes a seat frame 71, an armrest bracket 75, a link 81, a connection joint 85, an armrest driver 91, and an operation lever 95 as shown in FIGS. 6 and 7.

The seat frame 71 has a rod shape with a bent center region and is provided on one side of the seat 30.

One end of the seat frame 71 is supported by the bottom of the seat 30, and the other end protrudes to one side of the seat 30 to a position higher than the seat 30 without interfering with the seat 30.

The armrest bracket 75 is connected to one side of the bottom of the armrest 60. The armrest bracket 75 is connected by a hinge member 77, for example, a step hinge that rotates by a certain angle in stages as the hinge member 77, so that the armrest 60 may be tilted.

The seat frame 71 is connected to one end of the link 81, and the armrest bracket 75 is connected to the other end, so that the seat frame 71 and the armrest bracket 75 are interconnected.

One end of the link 81 is swivellably connected to the seat frame 71 by the connection joint 85 and is simultaneously provided to be liftable along the inside of the connection joint 85.

Accordingly, the link 81 has a rotation radius corresponding to the interval between one end of the link 81 and the other end of the link 81, for example, the interval between the connection joint 85 and the armrest bracket 75, and the link 81 swivels around the connection joint 85 and lifts and lowers along the inside of the connection joint 85.

The armrest driver 91 is supported on the armrest bracket 75 to lift and lower the link 81 relative to the seat frame 71.

Such an armrest driver 91 is provided to be inclined upward while forming an acute angle with respect to the bottom surface of the armrest 60.

Here, as the armrest driver 91, a common gas spring in which a spring is accommodated inside a cylinder containing compressed gas, and the spring automatically provides restoring force and compressive force by compressing the gas inside the cylinder, for example, using a force by which the spring is automatically compressed or expanded by gas pressure, may be applied. Accordingly, according to the operation of the operation lever 95 for manipulating the operation of the gas spring, the armrest 60 can be raised or lowered using the restoring force or compressive force of the spring generated as the spring expands or compresses.

Accordingly, in the case that the operation lever 95 is manipulated so that the force generated by the armrest driver 91 is applied toward the upper side of the armrest 60, the link 81 rises along the inside of the connection joint 85, and the armrest 60 rises to a certain height relative to the seat 30.

Conversely, in the case that the operation lever 95 is manipulated so that the force generated by the armrest driver 91 is applied toward the lower side of the armrest 60, the link 81 descends along the inside of the connection joint 85, and the armrest 60 descends to a certain height relative to the seat 30.

In addition, when the surgeon pushes the armrest 60 toward the rear of the surgical chair 1 for microscopy, the link 81 swivels around the connection joint 85 toward the rear of the surgical chair 1 for microscopy, so that the armrest 60 is located at the rear of the surgical chair 1 for microscopy.

Conversely, when the surgeon pulls the armrest 60 toward the front of the surgical chair 1 for microscopy, the link 81 swivels around the connection joint 85 toward the front of the surgical chair 1 for microscopy, so that the armrest 60 is located at the front of the surgical chair 1 for microscopy.

In addition, when the surgeon tilts the armrest 60 relative to the horizontal, the armrest 60 may be tilted at a certain angle in the direction in which the force is applied by the hinge member 77.

In this way, by adjusting the armrest 60 according to the surgical situation and the height of the surgeon's arm, fatigue of the shoulders and neck can be reduced, and accurate surgery can be performed.

In addition, by adjusting the angle of the armrest 60 according to the angle of the arm, the burden on the elbows and wrists can be reduced and a stable posture can be maintained.

As such, by adjusting the armrest 60 to be swivellable, liftable, and tiltable, surgical space is secured, joint mobility is improved, convenience of surgical tool manipulation is improved, surgeon fatigue is reduced, and surgical concentration can be improved.

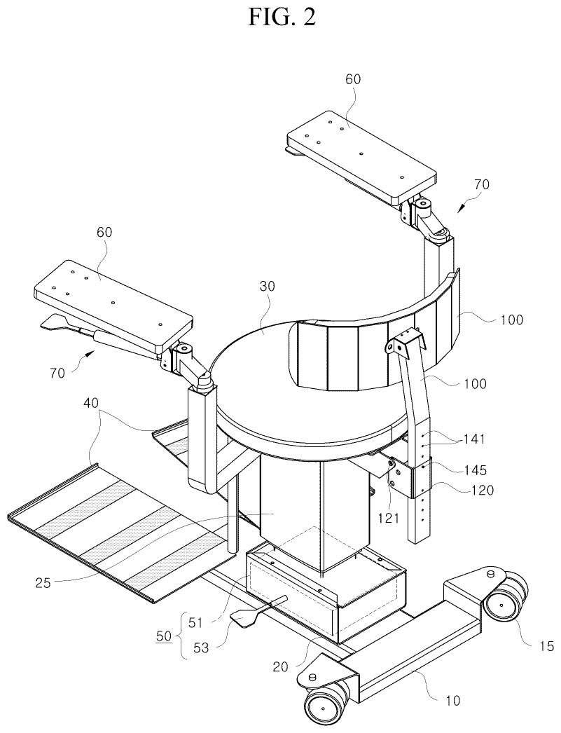

In addition, as shown in FIG. 2, the surgical chair 1 for microscopy according to an embodiment of the present disclosure may include a backrest 100 configured to support the back of a surgeon sitting on the seat 30, a backrest frame 110 supporting the backrest 100, a backrest bracket 120 to which the backrest frame 110 is coupled, and a seat bracket 130 connecting the backrest bracket 120 and the seat 30.

The backrest frame 110 has a bar shape of a certain length and is provided on the rear surface of the backrest 100 to be disposed upright relative to the seat 30.

The backrest bracket 120 and the seat bracket 130 are rotatably coupled by the backrest 100, and a torsion spring (not shown) that generates elastic force in the backrest bracket 120 may be provided around the backrest 100.

Accordingly, in the case that force is applied to the backrest 100 toward the rear of the seat 30 while the backrest 100 is disposed vertically relative to the seat 30, the backrest bracket 120 tilts around the backrest 100, so that the backrest 100 tilts relative to the seat 30, and in this case, the torsion spring is tensioned. Conversely, when the force applied toward the rear of the seat 30 is removed from the backrest 100, the torsion spring is compressed and the backrest 100 is restored to its original position by the elastic restoring force of the torsion spring.

In this way, by adjusting the angle of the backrest 100, the burden on the waist is minimized in accordance with the surgeon's spinal curve, and surgery can be performed in a comfortable posture.

Here, although not shown, a lumbar support configured to support the surgeon's lumbar spine may be provided on the backrest frame.

In addition, the backrest 100 of the surgical chair 1 for microscopy according to an embodiment of the present disclosure may include a plurality of coupling holes 141 formed at intervals along the longitudinal direction of the backrest frame 110, and a coupling protrusion 145 provided on the backrest frame 110 and coupled to any one of the plurality of coupling holes 141.

Here, the coupling hole 141 may be formed by being recessed in a hemispherical cross-sectional shape, and the coupling protrusion 145 may have a cross-sectional shape and size corresponding to the coupling hole 141 and may be formed to protrude toward the backrest frame 110.

Accordingly, by coupling the coupling protrusion 145 to any one of the plurality of coupling holes 141 formed at intervals along the longitudinal direction of the backrest frame 110, the height of the backrest 100 relative to the seat 30 can be adjusted while lifting and lowering the backrest frame 110 along the backrest bracket 120.

Accordingly, by adjusting the height of the backrest 100 according to the surgical situation and the surgeon's sitting height, the lower back and back can be effectively supported and surgery can be performed while maintaining a stable posture.

As described above, according to the present disclosure, it is possible to sit in a desired posture according to surgical situations and physical conditions, thereby performing surgery in a stable posture to improve surgical convenience, reduce surgeon fatigue, and enhance surgical concentration.

Meanwhile, in the above-described embodiment, the present disclosure has been described as being applied to a surgical chair for microscopy as an embodiment, but is not limited thereto, and the technical idea according to the present disclosure may be applied to a chair having a seat, armrests, a backrest, and footrests.

Although the embodiments of the present disclosure have been described with reference to the accompanying drawings, those skilled in the art will understand that the present disclosure may be implemented in other specific forms without changing its technical spirit or essential features. Therefore, the embodiments described above should be understood as illustrative in all respects and not restrictive.

What is claimed is:

1. A surgical chair for microscopy, comprising:
a base;
a housing mounted on an upper portion of the base;
a lifting post installed inside the housing;
a seat coupled to an upper portion of the lifting post and configured to allow a user to sit thereon;
a pair of footrests on which both feet of the user are placed;
a footrest connection frame connecting the seat and the footrests; and
a seat lifting module configured to lift and lower the lifting post to simultaneously lift and lower the seat and the pair of footrests.

2. The surgical chair of claim 1, wherein the seat lifting module comprises:
a seat lifting driver disposed inside the housing to lift and lower the lifting post; and
an operation pedal installed on the base to operate the seat lifting driver.

3. The surgical chair of claim 1, wherein the footrests are rotatably coupled to the footrest connection frame such that an arrangement angle of the footrests relative to the seat is adjusted.

4. The surgical chair of claim 1, further comprising:
a pair of armrests on which both arms are placed; and
an armrest adjuster configured to swivel, lift, and tilt the armrests relative to the seat.

5. The surgical chair of claim 4, wherein the armrest adjuster comprises:
a seat frame supported by the seat;
an armrest bracket to which the armrests are tiltably coupled by a hinge member;
a link having one end connected to the seat frame and another end connected to the armrest bracket;
a connection joint provided on the seat frame, and the link being coupled to the connection joint to be swivellable and liftable;
an armrest driver supported on the armrest bracket to lift and lower the link relative to the seat frame; and
an operation lever configured to manipulate an operation of the armrest driver.

6. The surgical chair of claim 1, further comprising:
a backrest configured to support a back of a surgeon sitting on the seat;
a backrest frame supporting the backrest;
a backrest bracket to which the backrest frame is coupled; and
a seat bracket connecting the backrest bracket and the seat.

7. The surgical chair of claim 6, further comprising a hinge pin rotatably coupling the backrest bracket and the seat bracket such that the backrest is tiltable relative to the seat.

8. The surgical chair of claim 6, further comprising:
a plurality of coupling holes formed at intervals along a longitudinal direction of the backrest frame; and
a coupling protrusion provided on the backrest bracket and configured to be coupled to any one of the plurality of coupling holes,
wherein a height of the backrest is adjustable relative to the seat.

9. The surgical chair of claim 1, further comprising an anti-slip pad disposed on the footrests to prevent slipping.

10. The surgical chair of claim 1, further comprising a plurality of casters rollably provided on the base to move the base along a floor.
